(19) United States
(12) Patent Application Publication
(10) Pub. No.: US 2020/0197287 A1
(43) Pub. Date: Jun. 25, 2020

(54) NEW HAIR BOTOX MATERIAL AND THE METHOD TO APPLY THIS MATERIAL TO HAIR

(71) Applicant: Sabahattin CETINTAS, Isparta (TR)

(72) Inventor: Sabahattin CETINTAS, Isparta (TR)

(21) Appl. No.: 16/640,739

(22) PCT Filed: Aug. 17, 2018
(86) PCT No.: PCT/TR2018/000090

§ 371 Date: Feb. 21, 2020

(30) Foreign Application Priority Data
Aug. 23, 2017 (TR) 2017/12588
Aug. 16, 2018 (TR) 2018/11969

(51) Int. Cl. A61K 8/65; A61Q 5/12

(52) U.S. Cl. CPC A61K 8/65; A61Q 5/12

(57) ABSTRACT

A hair botox contains, as a weight ratio, 5% to 30% of keratin in a weight of 100 g of water. This ratio is preferred as being between 15% and 30% keratin for excessively worn hair, and between 5% and 15% keratin for less worn hair. In the invention, in addition to 5%-30% keratin in 100 g of water, if desired, vitamins, minerals, herbal extracts, other proteins, other than keratin, and materials used in the cosmetic field may be used in a rate of 0.001% to 20% each.

### CROSS REFERENCE TO THE RELATED APPLICATIONS

This application is the national phase entry of International Application No. PCT/TR2018/000090, filed on Aug. 17, 2018, which is based upon and claims priority to Turkish Patent Application No. 2017/12588, filed on Aug. 23, 2017 and Turkish Patent Application No. 2018/11969, filed on Aug. 16, 2018, the entire contents of which are incorporated herein by reference.

### TECHNICAL FIELD

This invention includes an improvement on hair botox. The invention is concerned with the development of a new hair care and repair material particularly with hair botox and a new method of application of this material to hair. A much longer lasting and more effective hair care and repair operation can be realized through the invention in question.

### BACKGROUND

Today, hair care got a bigger importance than it was in the past. Many people, especially women, attach more importance to hair care and apply various chemical or biological cures for this.

The main reason for hair care's gaining importance is; the hair is substantially damaged and worn out especially because of the operations such as hair coloring or shaping in women in addition to the importance given to personal care in the developing society. When worn out hair specimens are examined in detail through an electronic microscope (SEM) in a laboratory environment, it is often seen that the hair follicles are worn out, or that the hair fibers are broken, or that the hair is broken.

In the known status of technique, it can be seen that many materials or methods are applied for hair care and especially for hair repair. It is observed that especially such products as mask, cream, serum, hair botox, shampoo, among them, are frequently used. However, the effect of such products on hair can usually last up to 1-2 weeks, and later, they completely lose their efficiencies. It is known that the same problems continue again. Furthermore, most of the cares made with these methods and materials can be applied in a hairdressers in a very troublesome, costly and time consuming manner. Since almost all of the methods and products mentioned above include chemical and synthetic materials, or high temperature and pressure is applied on hair, this very important occasion that the hair is worn more should be kept in mind.

In the known status of the technique, it is known that the material of keratin is used for hair care and repair in. Keratin, known as a protein type, is actually a hair care and repair material that has long been known. However, most of these keratin-related methods of use are in the shape of use in hair care products such as shampoos, creams, masks, and serum with chemical and synthetic ingredients. However, even though it is claimed that such keratinized products increase the benefits of the product, there are also increasing claims that it is not much different from other non-keratinized products and does not add any extra benefits for hair.

In the use of keratin as a hair care ingredient in the market, although, at first stage, an image that this product used is enriched with keratin and is more useful for hair, such synthetic and chemical additives completely eliminate the beneficial effects of keratin. Regarding this, the American patent with U.S. Pat. No. 2,434,688 mentions about hair care using keratin.

Another patent that is seen in the known status of the technique is the American patent with U.S. Pat. No. 4,279,996. In this patent, a method of obtaining a water-soluble keratin and the application of the material obtained by this method to hair is mentioned.

Another patent that is seen in the known status of the technique is the American patent with U.S. Pat. No. 7,189,268. In this patent, a keratin-based (keratin containing) bleaching agent is mentioned.

Another patent that is seen in the known status of the technique is the Europe patent with no. EP2422763. In this patent, a hair care product containing keratin together with a lot of chemicals is mentioned.

As it can be seen in the known status of the technique, keratin protein is a matter that has been known and used in hair care for almost a hundred years. However, the biggest problem with keratin in the known status of technique is that when keratin is used in combination with many chemical and synthetic materials, the keratin proteins cannot reach directly to the damaged parts of the hair, and as a result, it completely loses its effects and thus a real care and repair cannot be made for the hair. Even many chemical materials, which are claimed to be hair care and repair products, and containing keratin, damage the hair, and also prevent the keratin material from showing its real benefit for the hair.

### SUMMARY

Through the subject of the invention, hair botox (keratin based material application to the hair) material, a content which can perform hair care, repair and protection process in real sense and, with this content, a permanent hair care, repair and protection are targeted by developing a new method of application which has not been known until now.

In the method in which the keratin is used in liquid form, when keratin protein (the one used in the invention is hydrolyzed keratin) is applied directly to the damaged parts of the hair, the keratin proteins are enabled to be dry after the evaporation of the water that provides the solution, and they unite with the hair like bone, repair and fill the hair surface, making the hair smooth and shiny. In addition, these benefits, which it brings for the hair, will never lose later unlike as in synthetic and chemical materials or other methods.

The basic feature of the subject of the invention, the hair botox, is to mix water with keratin in certain rates and to apply it directly to the hair. And another basic is that this mixing ratio is diluted or has intense consistency according to the wear degree of, and structure of the hair, therefore, that a keratin-based material which is easily applied in many types of hair, and which provides more efficient repair can be obtained. This keratin-based material is called hair botox as it can be used in the parts. It is possible that higher keratin protein rates (rates will be given below) and more frequent applications can be applied in highly worn hair and much the same, less keratin use (diluted) and fewer applications can be applied in less worn hair.

Another advantage of the subject of the invention, hair botox, is that it achieves a permanent hair care with a very easy application without the need to heat and press the hair with a variety of devices and without the risk of damage to the hair as being in the known status of the technique.

On the other hand, since no chemical and synthetic materials are used with this hair botox, it can be used safely without any damage to the skin and hair.

With the subject of the invention, hair botox material, and a new application method of this hair botox to the hair, the keratin material treats the damaged hair surfaces and makes the hair smoother and thus helps to obtain brighter, more vivid and permanent protection. Therefore, the keratin material repairs the hair as well as providing permanent brightness, vividness and voluminous appearance which are temporarily brought by synthetic materials.

As a result of the subject of the invention, hair botox material, and a new application method of this hair botox to the hair, there is no need troublesome hair care, costly and time consuming operations. On the other hand, in the subject of the invention, hair botox, it is possible to use, according to the need of hair, various vitamins, extracts, minerals, proteins, cosmetics or natural or herbal and similar substances which can enrich the formula and increase its benefits.

### DETAILED DESCRIPTION OF THE EMBODIMENTS

The subject of the invention, hair botox, becomes applicable to the hair by being basically produced with water, preferable with pure water, in the rates to be stated below, in light dilution or intense consistency forms. In order to increase the effectiveness in the hair, there may also be a type and amount of usage which is also recommended according to the condition of the person and the hair. On the other hand, the hair botox in question; when it is produced with vitamins, extracts, minerals, proteins and other cosmetics that are necessary for hair care, it can also gain other useful features such as a separate protection and hair feeding and better hair care.

In the known status of the technique, the term of Hair Botox is the form of facial skin applied to the hair with keratin. For removing the wrinkles in the face, useful materials are injected to the skin using the botox method, just same as injecting keratin proteins to the worn, stressed and broken hair surface and enabling the hair to become smoother, shiny and more voluminous and healthier, and thus to have a better appearance.

Since the keratin protein constituting subject of the invention, hair botox, is mainly diluted with water, the term of liquid is used. Whereas this term is used as “liquid” in English language, the term of “liquid keratin” can be used as it is also used in Turkish for technical use. However, it is also known that it is called as hydrolyzed keratin in the cosmetic sector.

As the weight ratio of the subject of the invention, hair botox, is basically as a proportion in 100 g of water; it contains between 5% and 30% keratin. This rate is preferred as being 15%-30% for excessively worn hair, and 5%-15% keratin for less worn hair. In the invention, in addition to 5% to 30% keratin in 100 g of water, if desired, vitamins, minerals, extracts, proteins and other cosmetic ingredients, other than keratin, may be used in a rate of 0.001% to 20% each.

Although keratin in the content of the subject of the invention, hair botox, is used as liquid under normal conditions, if desired, it is also possible to use it in the form of a spray in suitable pressurized or non-pressurized containers.

It is necessary to apply the following steps in order so that the subject of the invention, hair botox, can be successful.

Purification of the Hair (100)

Before applying the subject of the invention, hair botox, it is necessary to wash the hair two or three times with hot water and preferably with a cleansing shampoo or any cleaner, containing no synthetic material and silicone, thus to enable hair follicles to be spaced.

It is necessary that any permanent hair care and especially herbal hair care oils which prevent keratin binding and decrease its effect not be used at least one week prior to application of the subject of the invention, hair botox.

Drying the Hair (200)

The washed hair should be dried or slightly moistened with a towel or dryer. The subject of the invention, hair botox, can also be applied to wet hair according to the need.

Rubbing Hair Botox in the Hair (300)

Keratins should be rubbed in the dry or slightly moistened hair (wet hair, if needed) with hand or brush or using a spray bottle, by massaging. In order to dry the hair, it must be kept open without tying or it must be completely dried with 5-10 minutes intervals and preferably with the help of warm blow to dry faster.

During waiting or drying, the hair may temporarily and sometimes give a feeling like sticky to each other and hard.

In this specified operation, the hair is not combed. This problem is easily solved by rinsing directly with plenty of warm water and by treating with appropriate products such as mask, cream, serum, which are most suitable for hair structure.

Rinsing the Dried Hair (400)

Liquid keratin loaded and dried hair should be rinsed several times preferably with warm water. During rinsing, the hair may temporarily become hard and tangled. For this case, the desired comfort is achieved by using products such as masks, creams or hair serum which are most suitable for hair structure.

A New Application for Excessively Worn Hair;

It is the process of making the second hair botox for excessively worn hair. It is the performance of the second session, without washing the hair, when the hair is in slightly moist and dry condition after the first keratin load in cares need to be repeated in certain days or intervals in order to prevent people from spending too much time and being busy or to make it easier in the hairdressing salons. The most important advantage of this application is that the application can be repeated and a faster care and repair can be performed without having to wash the hair again and without being have to wait for a few days. Therefore, care that lasts days with certain periods of time can be easily accomplished within a few hours.

Application on Sensitive Hair and Hair about to Break;

The method to be applied on Sensitive hair and Hair about to break is slightly more different. It is rarely observed that when people tie their hair wet and go to bed with wet hair after keratin loading, the hair stick together after drying and some breaking problems occur while brushing the hair. As a solution to this situation, with not tying the wet hair or not going to bed when the hair is wet, hair's sticking and breaking while combing can be prevented. Moreover, it is recommended that the hair be washed with plenty of warm water before brushing the hair in the case of sticking one another while it is getting dried, and then that care be done with the help of products such as a mask, cream serum which are most suitable for the structure of the hair.

It contains the use of appropriate care products for softening and easy brushing the rinsed hair in the subject of the invention, hair botox material and its method.

The subject of the invention, hair botox, repair worn and dropping, broken, hard combed, thinned, hard and mated hair, and, at the same time, brings the hair into more vivid, shiny, voluminous, smoother and stronger structure. According to the known status of the technique, cares made with other keratin-containing products and all these benefits provided for the hair completely lost their effects in the short time due to frequent washing of the hair and its presence in the sea or pool water environment. The biggest difference and superiority of hair botox is that it can enable all of this beneficial hair care and repair to be accomplished permanently.

Application Form of the Invention to the Industry

The subject of the invention, hair botox, is a product which can be used both in the hairdresser environment and easily applied to their hair by people in their home by themselves. Due to the lack of chemical content and the natural mixture formed with water in the application of the hair botox, especially, it poses no threat to the health of pregnant and breastfeeding women and young aged-children, and it gives no harm to the skin and body and can be used safely. Despite being used without getting help from specialists, it does not create any health risks, and more importantly it does not lose its permanent effect under no circumstances.

The subject of the invention, hair botox, can be produced as a liquid in bottles or in the form of spray in suitable pressure or non-pressure vessels and can be applied to any hair.

It can be easily diluted with water afterwards by the user in the case of being produced in an intense consistency, and also provides ease of use in special proportions peculiar to the users.

The keratin protein can, when desired, be sold while it is in powder form and can be made liquefied and become useful at certain rates.

What is claimed is:

1. A hair botox, comprising keratin and water at a ratio of 5-30:100 by weight.

2. The hair botox according to claim 1, wherein the hair botox comprising the keratin and water at a ratio of 15-30:100 by weight is for excessively worn hair, and the hair botox comprising the keratin and water at a ratio of 5-15:100 by weight is for less worn hair.

3. The hair botox according to claim 1, further comprising 0.001%-20% of each of proteins, vitamins, minerals, and extracts by weight.

4. A method for using the hair botox according to claim 1, comprising the following steps:
purifying hair,
first drying the hair,
rubbing the hair botox in the hair and
rinsing the hair after second drying the hair.

5. The method according to claim 4, wherein during purifying the hair before applying the hair botox, a step of washing the hair two or three times with hot water and a cleaner without synthetic material and silicone is included.

6. The method according to claim 4, wherein a step of drying the hair by a towel or dryer is included in the step of first drying the hair to make the hair dry or moisturized.

7. The method according to claim 4, wherein the hair botox is rubbed in the hair after being dried or moistened with a plastic gloved hand by massaging for 5-10 minutes, and then the hair is dried naturally or is dried with a warm blow in the step of rubbing the hair botox in the hair.

8. The method according to claim 4, wherein a step of rinsing the hair having absorbed liquid keratin with warm water in the step of rinsing the hair after second drying.

9. The method according to claim 4, further comprising a step of performing a second hair botox treatment, wherein the hair is excessively worn hair, the second hair botox treatment comprises rubbing the hair botox in the hair without washing the hair when the hair is in a damp or dry condition.

10. The method according to claim 4, wherein when a user does not tie the hair and goes to bed when the hair is wet, the hair gets sticky after drying and breaking problems occur while combing, the hair's sticking and breaking during combing is prevented due to not tying the wet hair or not going to bed when the hair is wet.

11. The method according to claim 4, wherein a step of using care products for softening and brushing is included in the step of rinsing the hair after drying.

12. The hair botox according to claim 2, further comprising 0.001%-20% of each of proteins, vitamins, minerals, and extracts by weight.
